(19) United States
(12) Patent Application Publication
(10) Pub. No.: US 2025/0339104 A1
(43) Pub. Date: Nov. 6, 2025

(54) ARTIFICIAL INTELLIGENCE SYSTEM FOR FORECASTING NEAR-TERM SUDDEN CARDIAC DEATH AND ADVERSE CARDIOVASCULAR EVENTS

(71) Applicant: Morteza Naghavi, Long Beach, CA (US)

(72) Inventor: Morteza Naghavi, Long Beach, CA (US)

(21) Appl. No.: 18/652,123

(22) Filed: May 1, 2024

(51) Int. Cl. A61B 5/00; A61B 5/0205; A61B 5/332; A61B 5/333; A61B 6/00; G16H 50/20

(52) U.S. Cl. CPC A61B 5/7275; A61B 5/0205; A61B 5/7267; G16H 50/20; A61B 5/332; A61B 5/333; A61B 6/5217

(57) ABSTRACT

According to an aspect of the present invention, there is provided computer-implemented method for forecasting near-term sudden cardiovascular events, comprising: pretraining a large language model transformer architecture using a processor with an associated computer memory device to recognize text associated with sudden cardiovascular events cases reporting sudden cardiovascular events following a medical exam appointment; obtaining relevant data regarding an asymptomatic individual comprising one or more of a coronary artery calcium (CAC) scan, a coronary CT angiography (CCTA), chest CT, blood markers, and electrocardiogram; providing the relevant data of the asymptomatic individual to the trained computer-implemented artificial neural network; receiving a forecast of the chance of near-term sudden cardiovascular events in the asymptomatic individual; and recommending the next diagnostic or therapeutic step for the asymptomatic individual.

### BACKGROUND

Sudden cardiac death is the most common and often the first manifestation of coronary heart disease and is responsible for ˜50% of the mortality from cardiovascular disease in the United States and other developed countries. The sudden onset of this condition in previously asymptomatic patients is tragic and devastating to many families. Solutions which more accurately predict such events are urgently needed.

A major shortcoming in preventive cardiology is forecasting sudden cardiac death and other adverse CVD events in asymptomatic individuals as currently no tool exists to predict such events in the near future. The only available test is a 10-year risk estimator based on CVD risk factors which does not work at an individual level but rather as a population-based epidemiological predictor.

### SUMMARY OF INVENTION

The present invention is inspired by current forecasting models for short-term prediction of weather which has been customarily used for providing alerts for severe weather events and percentage chances of particular weather phenomena like rain. A similar approach is taken for heart attacks and other sudden cardiovascular events. Just as with computerized weather prediction systems, a number of variables are considered by the methods and systems of the present invention to generate a forecast of sudden cardiovascular events. The present invention provides a solution to an unmet problem of forecasting sudden cardiovascular events. The Framingham heart study and its progeny typically predict chances of cardiovascular events over long periods of time such as 10 years or lifetime. The American Heart Association PREVENT score is based on a similar timeframe of 10 years. The present invention teaches a method which predicts with the granularity needed to forecast for much shorter periods.

Accordingly, the present invention uses a large language model to identify a unique dataset of cases involving sudden cardiac death among asymptomatic individuals who visited a clinic provided blood sample and underwent medical imaging and clinical evaluation shortly before experiencing a CVD adverse event leading to sudden cardiac death. It leverages this unique dataset to provide methods and systems for AI-enabled event forecasting specific to these unexpected occurrences of sudden cardiac death due to major cardiovascular events among asymptomatic individuals. Such an AI predictor will have the ability for pattern recognition of high risk combination for a near future adverse event.

According to an aspect of the present invention, there is provided computer-implemented method for forecasting near-term sudden cardiovascular events, comprising: pretraining a large language model transformer architecture using a processor with an associated computer memory device to recognize text associated with sudden cardiovascular events cases reporting sudden cardiovascular events following a medical exam appointment at which one or more of a coronary artery calcium (CAC) scan, a coronary CT angiography (CCTA), and chest CT, a blood sample, an electrocardiogram were obtained; collecting, using the trained large language model, cases reporting sudden cardiovascular events following a medical exam appointment at which one or more of a coronary artery calcium (CAC) scan, a coronary CT angiography (CCTA), and chest CT, a blood sample, an electrocardiogram were obtained; storing in a database on a computer memory device the obtained coronary artery calcium (CAC) scans, a coronary CT angiographies (CCTA), chest CT, blood markers, and electrocardiograms; storing in the database coronary artery calcium (CAC) scans, a coronary CT angiographies (CCTA), and chest CT, blood markers, and electrocardiograms for control cases of patients not experiencing sudden cardiovascular events following a cardiologist appointment; training a computer-implemented artificial neural network based on the coronary artery calcium (CAC) scans, a coronary CT angiographies (CCTA), chest CT, blood markers, and electrocardiograms of the collected cases and the control cases; obtaining relevant data regarding an asymptomatic individual comprising one or more of a coronary artery calcium (CAC) scan, a coronary CT angiography (CCTA), chest CT, blood markers, and electrocardiogram; providing the relevant data of the asymptomatic individual to the trained computer-implemented artificial neural network; receiving a forecast of the chance of near-term sudden cardiovascular events in the asymptomatic individual; and recommending the next diagnostic or therapeutic step for the asymptomatic individual.

### BRIEF DESCRIPTION OF THE DRAWINGS

FIG. 1 illustrates two cases in which patients suffered near-term adverse cardiovascular events.

### DETAILED DESCRIPTION

Cardiovascular Disease (CVD) has been the #1 cause of death and healthcare costs in the US for decades. Every year over 600,000 first-time heart attacks unexpectedly hit asymptomatic Americans. Even though, the awareness on CVD risk factors is above 95% meaning almost all US adults are aware of the risk associated with these risk factors, currently less than 3% of US adults aged 20-79 years have an optimal cardiovascular risk factors profile defined as: total cholesterol<200 mg/dL (5.17 mmol/L), blood pressure<120/<80 mm Hg, non-smoker, body mass index (BMI)<25 kg/m2, fasting plasma glucose<100 mg/dL (5.56 mmol/L). Clearly, new strategies are needed.

The present invention aims to use AI to detect who will have a cardiovascular event, such as sudden cardiac death, heart attack, or stroke, within a year (detect the Vulnerable Patient), in hopes that interventions can stave off such events.

The first step is to identify cases of sudden cardiac death for their predictive value with respect to currently asymptomatic individuals. To do so, a large language model is proposed to detect cases in which sudden cardiac death has occurred in individuals within a short time after data points such as coronary artery calcium (CAC) scans, a coronary CT angiographies (CCTA), and chest CT inflammatory signaling molecules, metabolomic markers, and proteomics were collected for the individuals. An LLM base model (such as Meta's Llama 2) can be used, which is typically tables of vector weights analyzed with software technologies like TensorFlow or PyTorch, and run on hardware that can include various NVIDIA graphical processor units that implement NVIDIA's CUDA (Compute Unified Device Architecture).

A vast and varied dataset from medical journals, news articles, public records, the internet and other sources is assembled including potentially billions of text sources. This can include books, articles, websites, death records, and more. The data is preprocessed by tokenizing the text into smaller units (words or subwords), removing irrelevant content, and organizing it for efficient training.

The model is initiated with random weights, creating a blank slate for learning. These weights will be adjusted during the training process through backpropagation.

Next a pre-training objective is defined, typically a language modeling task. The model learns to predict the next word in a sentence related to cases describing sudden cardiac death, given the preceding words, fostering an understanding of syntax, grammar, and context.

After cases have been collected, in a second step of the process of embodiments of the present invention pre-processed data is fed into the model and the model for predicting sudden cardiac death is trained using, for example, gradient-based optimization techniques like stochastic gradient descent. The model's parameters are updated iteratively to minimize the difference between predicted and actual next words.

Model size is a critical consideration. Larger models can capture more intricate patterns but require more computational resources. Balancing computational capacity with model size is essential.

In an alignment phase, the model for predicting sudden cardiac death is adapted to specific tasks through fine-tuning on task-specific data. This involves continuous training of the model using the task-specific dataset while keeping the weights learned during pre-training frozen. This allows the model to retain its general language understanding while adapting to the task-specific data related to prediction of sudden cardiac death.

Unlike the report of Gustafsson, S et al that short-term risk prediction was attained by serum biomarkers only, the subject matter of this invention requires a combination of risk assessment based on imaging and non-imaging data. Without the imaging data, the accuracy of such a short-term risk predictor model can be hampered. Gustafsson, S., Lampa, E., Jensevik Eriksson, K. et al. Markers of imminent myocardial infarction. Nat Cardiovasc Res 3, 130-139 (2024).

In an embodiment of the present invention, an implantable ECG loop recorder alerts patients and providers on dangerous electrical signs of a CVD event.

Yet in another embodiment, the wearable monitor is continuous monitoring of a serum or blood biomarker that alerts patients and providers on increased levels of the biomarker for a CVD event.

Yet in another embodiment, the wearable monitor is a combined ECG and biomarker monitor that alerts patients and providers when certain thresholds are met.

The present invention thus provides system of developing such a CVD risk forecaster by selecting high risk cases based on their imaging findings and clinical risk factors and having them wear the continuous ECG and serum biomarker monitor to be able to collect sufficient data in these high-risk cases prior to a sudden adverse event.

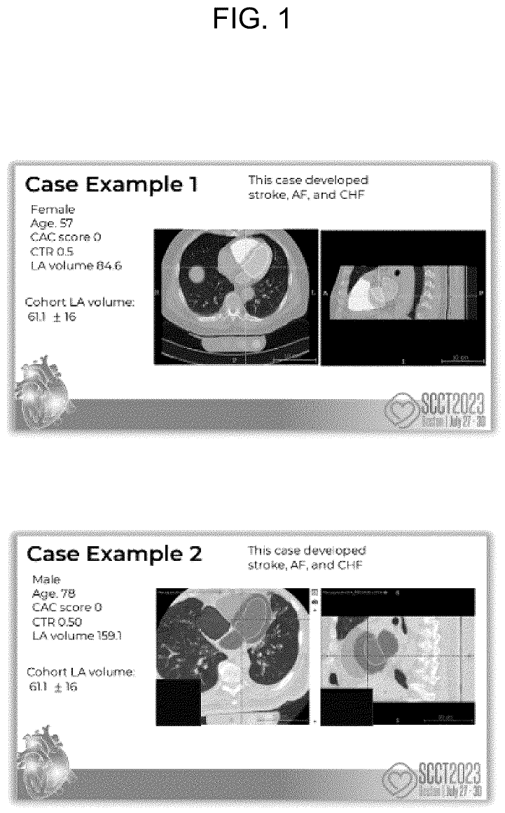

FIG. 1 illustrates two cases in which patients suffered near-term adverse cardiovascular events.

Case 1 is a 57-year old female and case 2 is a 78-year old male. Both suffered near-term adverse cardiovascular events comprising stroke, AF, and CHF.

The cases are used by the AI as examples of cases in which individuals suffered near-term adverse cardiovascular events. By providing a large number of cases of these cases and cases where there were no near-term adverse cardiovascular events, AI can assess the differences and apply them to a particular patient's case.

The AI is trained based on a rare collection of existing data from numerous longitudinal studies throughout US, Europe, and South America. Each study contributes a unique set of data from asymptomatic individuals who shortly (i.e. hours, days, or weeks, up to 12 months) after their medical visit had a sudden cardiac death or an adverse CVD event.

During the medical visit, they must have had a blood draw (banked), and their medical records must include a complete clinical evaluation along with a coronary artery calcium (CAC) scan, a coronary CT angiography (CCTA), or a chest CT scan obtained within 2 years prior to the event.

By applying deep learning techniques to this set of rare data, AI is trained to identify individuals at very high risk for a near-term event. For the first time, the field of cardiology will be able to predict who will have a heart attack, stroke, or an adverse CVD event within 12 months.

No longitudinal cohort or biobank in the world, by itself, has enough cases for this training. Hence, all prospective cardiovascular epidemiological studies worldwide are used and 14 large cohorts are included that amount to about 1,000 cases.

Since the pioneering Framingham Heart Study in 1960s introduced CVD risk factors, the practice of preventive cardiology has been based on long-term CVD risk prediction. Physicians tell their patients that based on their risk factors (age, gender, blood pressure, cholesterol, diabetes, smoking etc.) and their risk of developing cardiovascular disease in the next 10 years is X. The median and mean for X are 2.7% and 5.2% respectively.

Although such a long-term risk assessment is necessary, it is not enough. It does not trigger immediate preemptive actions and cannot detect asymptomatic patients who are vulnerable to a near-term CVD event.

A medical analogy would be finding a tumor in a cancer patient that gets serious attention and triggers immediate interventions to improve outcomes. Having such a predictive tool in cardiology can cause a paradigm shift resulting in developing new treatments. Developing this highly desired tool is the purpose of the present invention.

The present invention is able to develop algorithms and a Software as Medical Device (SaMD) that will provide a patient with a forecast of approximately 20% chance of near-term sudden cardiac death and adverse cardiovascular events in asymptomatic individuals with no history of cardiovascular disease (CVD) within the next 12 months. This is far more compelling and actionable than average 5.2% within the next 10 years.

It utilizes the Framingham Heart Study, MESA, ARIC, UK Biobank, HNR, Biolmage, and the Dallas Heart Study for AI training. External validation to test for discrimination and calibration is conducted using other longitudinal observational studies that provide adjudicated cardiovascular event information, such as MiHeart, JHS, DANRISK and ROBINSCA.

Additionally, AI is used to characterize individuals who, despite high conventional risk due to hyperlipidemia, hypertension, diabetes, smoking and obesity have lived over 80 years with no CHD events (the Invulnerable Patient).

The present invention allows for discovery of new targets for drug and possibly vaccine development. The AI algorithms can be used with additional data collected over time to increase AI's predictive value.

The present invention dramatically improves upon the current practice of preventive cardiology and provides opportunities not only for accurate risk assessment but also has the potential to yield new therapeutic targets including vaccine for heart attacks.

The AI tool of the present invention is a SaaS product therefore readily scalable to access worldwide.

Alerting the very high-risk individuals who have no symptoms and are completely unaware of their high risk of a catastrophic health event in the near future will likely cause immediately proactive and preemptive actions.

The primary health outcome will be significant reductions in sudden cardiac death, acute coronary syndromes, and cerebrovascular events.

This AI-enabled approach in preventive cardiology will be revolutionary and highly innovative. Nonetheless it is based on proven track record in cancer treatment where a detection of a malignant tumor can trigger rapid response and immediate compliance to intensive treatments such as chemotherapy.

Whereas today, preventive cardiology faces poor compliance with over half of statin prescriptions not filled after the first year.

The proposed AI-enabled solution can cause a paradigm shift in preventive cardiology and disrupt some of the current imprecise population-based risk assessment and therapeutic strategies with such high NNT (number needed to treat).

Instead, highly effective personalized preemptive therapies (coronary artery bypass graft) can be applied to a small number of very high-risk patients.

The proposed AI-enabled solution will impact millions of lives worldwide as CVD is the number 1 killer in most countries and kills over 17 million annually.

As shown in the modality table below, the quantitative jump from the existing solution to the proposed solution will be on the order of magnitude (approximately 40 times more precise). Furthermore, the AI algorithms will collect additional data over time and increase AI's predictive value.

The proposed solution will greatly enhance the accuracy of targeted therapy and will reduce unnecessary waste of healthcare resources. It will result in added millions of productive life years for human beings that will contribute to the world's economy.

As an illustrative example, HeartLung.AI's breakthrough AutoChamber™ AI Software as a Medical Device (SaMD) can detect high risk patients for heart failure and stroke. It uses deep learning algorithms to visualize chambers volume in non-contrast cardiac CT scans which human eyes cannot detect.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description.

Other embodiments may be utilized and derived from the present invention, such that structural and logical substitutions and changes may be made without departing from the scope of this disclosure.

Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments.

Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A computer-implemented method for forecasting near-term sudden cardiovascular events, comprising:
pretraining a large language model transformer architecture using a processor with an associated computer memory device to recognize text associated with sudden cardiovascular events cases reporting sudden cardiovascular events following a medical exam appointment at which one or more of a coronary artery calcium (CAC) scan, a coronary CT angiography (CCTA), and chest CT, a blood sample, an electrocardiogram were obtained;
collecting, using the trained large language model, cases reporting sudden cardiovascular events following a medical exam appointment at which one or more of a coronary artery calcium (CAC) scan, a coronary CT angiography (CCTA), and chest CT, a blood sample, an electrocardiogram were obtained;
storing in a database on a computer memory device the obtained coronary artery calcium (CAC) scans, a coronary CT angiographies (CCTA), chest CT, blood markers, and electrocardiograms;
storing in the database coronary artery calcium (CAC) scans, a coronary CT angiographies (CCTA), and chest CT, blood markers, and electrocardiograms for control cases of patients not experiencing sudden cardiovascular events following a cardiologist appointment;
training a computer-implemented artificial neural network based on the coronary artery calcium (CAC) scans, a coronary CT angiographies (CCTA), chest CT, blood markers, and electrocardiograms of the collected cases and the control cases;
obtaining relevant data regarding an asymptomatic individual comprising one or more of a coronary artery calcium (CAC) scan, a coronary CT angiography (CCTA), chest CT, blood markers, and electrocardiogram;
providing the relevant data of the asymptomatic individual to the trained computer-implemented artificial neural network;
receiving a forecast of the chance of near-term sudden cardiovascular events in the asymptomatic individual; and
recommending the next diagnostic or therapeutic step for the asymptomatic individual.

2. The method of claim 1, further comprising using a deep learning algorithms to visualize chambers volume in non-contrast cardiac CT scans which human eyes cannot detect.

3. The method of claim 1, further comprising using a metric related to a HEART Score to determine at least in part the therapy administered to the patient.

4. The method of claim 1, further comprising using a metric related to vasa vasorum density to further assess the risk and to determine at least in part the therapy administered to the patient.

5. The method of claim 1, further comprising preparing for optimizing coronary revascularization procedures based at least in part on the forecast.

6. A system configured to perform the method of claim 1.

7. The system of claim 6, wherein individuals categorized potentially as high risk based on one or more of a coronary artery calcium (CAC) scan, a coronary CT angiography (CCTA), chest CT, blood markers, and electrocardiogram are administered a wearable cardiovascular monitor that allows for continuously monitoring ECG and other markers of CVD event risk.

8. The system of claim 7, wherein the wearable monitor is an implantable ECG loop recorder that alerts patients and providers on dangerous electrical signs of a CVD event.

9. The system of claim 7, wherein the wearable monitor continuously monitors a serum or blood biomarker and alerts patients and providers on increased levels of the biomarker for a CVD event.

10. The system of claim 7, wherein the wearable monitor is a combined ECG and blood biomarker monitor that alerts patients and providers when certain thresholds are met.

11. The system of claim 7, wherein the wearable monitor continuously monitors physiological data such as heart rate, blood oxygenation, vascular resistance, and other hemodynamic markers.

12. A system of developing a sudden CVD event forecaster by selecting high risk cases based on their imaging findings and clinical risk factors and having them wear the continuous ECG and serum biomarker monitor to be able to collect sufficient data in these high-risk cases prior to a sudden adverse CVD event and by selecting low risk cases based on their imaging findings and clinical risk factors and having them wear the continuous ECG and serum biomarker monitor to be able to collect sufficient data in these low-risk cases who do not experience a sudden adverse CVD event, wherein training AI based on the data from the high risk cases and low risk cases enables the AI system to develop a sudden CVD event forecaster.
